(19) United States
(12) Patent Application Publication
(10) Pub. No.: US 2011/0295016 A1
(43) Pub. Date: Dec. 1, 2011

(54) A NEW PROCESS FOR PREPARING 4-[4-METHYL-5-(CL-10ALKYLTHIO/C5-10ARYL-CL-6ALKYLTHIO)-4H-1,2,4-TRIAZOL-3-YL]PYRIDINES

(75) Inventors: Hans Åström, Sodertalje (SE); Elfyn Jones, Sodertalje (SE); Tim Ståhlberg, Sodertalje (SE)

(73) Assignee: ASTRAZENECA AB, Sodertalje (SE)

(21) Appl. No.: 13/139,090

(22) PCT Filed: Dec. 11, 2009
(86) PCT No.: PCT/SE09/51404

§ 371 Date: Aug. 15, 2011

(30) Foreign Application Priority Data
Dec. 12, 2008 (US) 61/122044

(51) Int. Cl. C07D 401/04

(52) U.S. Cl. 546/272.4

(57) ABSTRACT

The invention relates to a method of manufacturing a compound according to formula (I) wherein R is C₁₋₆alkylor C₅₋₁₀aryl-C₁₋₆alkyl, comprising the steps of: a) reacting isonicotinohydrazide and methyl isothiocyanate, thereby obtaining 2-isoni-cotinoyl-N-methylhydrazinecarbothioamide; b) under alkaline conditions allowing said 2-isonicotinoyl-N-methylhydrazinecar-bothioamide to undergo a ring-forming reaction, thereby obtaining 4-methyl-5-pyridin-4-yl-2,4-dihydro-3H-1,2,4-triazole-3-thione; and c) under alkaline conditions allowing said 4-methyl-5-pyridin-4-yl-2,4-dihydro-3H-1,2,4-triazole-3-thione to react with R—X, wherein R has the same meaning as in formula I and X is selected from the group of Cl, Br and I, thereby obtaining a compound according to formula I; wherein steps a), b) and c) are carried out in an aqueous environment without intermediate isolations.

### FIELD OF THE INVENTION

The present invention relates to a new process for large-scale production of compounds chosen from the group of 4-[4-methyl-5-(C₁₋₁₀alkylthio)-4H-1,2,4-triazol-3-yl]pyridines and of 4-[4-methyl-5-(C₅₋₁₀aryl-C₁₋₆alkylthio)-4H-1,2,4-triazol-3-yl]pyridines. The invention also relates to new compounds produced by the method as well as using these compounds as intermediates for manufacturing pharmaceutically active larger compounds.

### TECHNICAL BACKGROUND

4-(5-{(1R)-1-[5-(3-chlorophenyl) isoxazol-3-yl]ethoxy}-4-methyl-4H-1,2,4-triazol-3-yl)pyridine is an antagonist of the mGluR5 receptor. Accordingly, this compound is expected is to be well suited for treatment of mGluR5-mediated disorders, such as acute and chronic neurological and psychiatric disorders, gastrointestinal disorders and chronic and acute pain disorders. This and similar compounds are disclosed in WO, A1, 2007/040982. This patent application also describes a process where 4-[4-methyl-5-(methylsulfonyl)-4H-1,2,4-triazol-3-yl]pyridine, an intermediate compound in the synthesis of 4-(5-{(1R)-1-[5-(3-chlorophenyl) isoxazol-3-yl]ethoxy}-4-methyl-4H-1,2,4-triazol-3-yl)pyridine, is manufactured in a four-step process.

The process of WO, A1, 2007/040982 is a process that is suitable for laboratory scale. Accordingly, there is a need for an improved process which is possible to carry out in larger scale, and which ideally is simple, cost effective, and without harmful impact on the environment.

### SUMMARY OF THE INVENTION

In one aspect, the invention provides a method of manufacturing a compound according to formula I

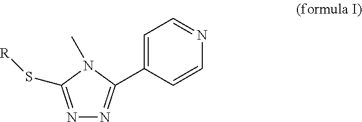

wherein
R is C₁₋₆ alkyl or C₅₋₁₀ aryl-C₁₋₆ alkyl.

The method comprises the steps of:

a) reacting isonicotinohydrazide and methyl isothiocyanate, thereby obtaining 2-isonicotinoyl-N-methylhydrazinecarbothioamide;
is b) under alkaline conditions allowing said 2-isonicotinoyl-N-methylhydrazinecarbothioamide to undergo a ring-forming reaction, thereby obtaining 4-methyl-5-pyridin-4-yl-2,4-dihydro-3H-1,2,4-triazole-3-thione; and
c) under alkaline conditions allowing said 4-methyl-5-pyridin-4-yl-2,4-dihydro-3H-1,2,4-triazole-3-thione to react with R—X, wherein R has the same meaning as in formula I and X is Cl, Br or I, thereby obtaining a compound according to formula I.

An essential feature of the method is that steps a), b) and c) are carried out in an aqueous environment without intermediate isolations.

In a preferred embodiment, the invention relates to a method for manufacturing a compound according to formula II

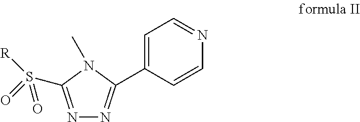

wherein
R has the same meaning as denoted above
comprising the steps of
i) carrying out the method of steps a), b) and c); and
ii) oxidizing said compound according to formula I, thereby obtaining a compound ici according to formula II.

In another aspect, the invention relates to intermediate compounds according to formula I

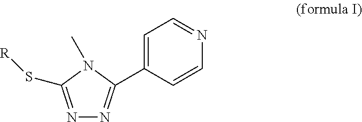

is wherein
R is C₂₋₆alkyl or C₅₋₁₀aryl-C₁₋₆alkyl,

### DETAILED DESCRIPTION OF THE INVENTION

The present invention provides a solution to the problem of providing a process suitable for large-scale production of intermediate compounds suitable in the synthesis of antagonists of the mGluR5 receptor, such as 4-(5-{(1R)-1-[5-(3-chlorophenyl) isoxazol-3-yl]ethoxy}-4-methyl-4H-1,2,4-triazol-3-yl)pyridine. The new process is simplified in comparison with prior art processes as no isolation or purification steps are required between the first three synthesis steps. Moreover, steps a)-c) are carried out in an aqueous environment preferably using NaOH or KOH as sole basic reagent. However, alternative bases may also be considered, e.g. amine bases such as trialkylamines where the alkyl may be C₁₋₆alkyl.

Accordingly, in a first aspect, the invention provides a method of manufacturing a compound according to formula I

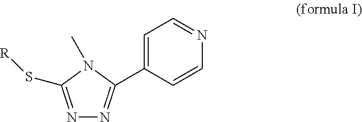

wherein
R is f C₁₋₁₀alkylor C₅₋₁₀aryl-C₁₋₆alkyl.

The method comprises the steps of:

a) reacting isonicotinohydrazide and methyl isothiocyanate, thereby obtaining
2-isonicotinoyl-N-methylhydrazinecarbothioamide;
b) under alkaline conditions allowing said 2-isonicotinoyl-N-methylhydrazinecarbothioamide to undergo a ring-forming reaction, thereby obtaining 4-methyl-5-pyridin-4-yl-2,4-dihydro-3H-1,2,4-triazole-3-thione; and
c) under alkaline conditions allowing said 4-methyl-5-pyridin-4-yl-2,4-dihydro-3H-1,2,4-triazole-3-thione to react with R—X, wherein R has the same meaning as in formula I and X is selected from the group of Cl, Br and I, thereby obtaining a compound according to formula I.

An essential feature of the method is that steps a), b) and c) are carried out in an aqueous environment without intermediate isolations. As disclosed herein, the term “aqueous environment” is intended to mean an environment mainly composed of water, such as a water solution of one or more water-soluble salts, or a mixture of water and one or more water-miscible organic solvents. Preferably, the aqueous environment is a water solution. As disclosed herein, the term C₁₋₆alkyl relates to a straight or branched alkyl group having 1, 2, 3, 4, 5 or 6 carbon atoms.

In this specification, unless stated otherwise, the term “alkyl” includes both straight and branched chain alkyl groups and may be methyl, ethyl, n-propyl, i-propyl, n-butyl, i-butyl, s-butyl, t-butyl, n-pentyl, i-pentyl, t-pentyl, neo-pentyl, n-hexyl, i-hexyl or t-hexyl. The term “C₁₋₃alkyl” refers to an alkyl group having 1, 2 or 3 carbon atoms, and may be methyl, ethyl, n-propyl or i-propyl.

As disclosed herein, the term “C₅₋₁₀aryl” refers to an optionally substituted monocyclic or bicyclic hydrocarbon ring system containing at least one unsaturated aromatic ring. Examples and suitable values of the term “aryl” are phenyl, naphthyl, 1,2,3,4-tetrahydronaphthyl, indyl and indenyl.

In a preferred embodiment, a single base selected from the group of NaOH and KOH is used. The base is added to step b). Alternatively, amine bases such as trialkylamines where the alkyl may be C₁₋₆alkyl, could be considered.

In a preferred embodiment, the product obtained in step c) is isolated by filtration.

In a second aspect, the invention provides a method of manufacturing a compound according to formula II

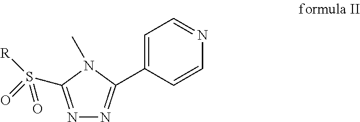

wherein
R has the same meaning as in formula II,
comprising the steps of
i) carrying out the method according to said first aspect; and
ii) oxidizing said compound according to formula I, thereby obtaining a compound according to formula II.

In a preferred embodiment, step ii) is carried out in an optionally acid aqueous solution of an oxidant, selected from the group of hydrogen peroxide, sodium permanganate, potassium permanganate, NaIO₄₅ KIO₄₅ potassium monopersulfate, NaBO₃ and KBO₃.

In a preferred embodiment, said acid aqueous solution is a sulfuric acid solution.

In a preferred embodiment, step ii) is carried out in presence of a catalytic amount of a tungstate, such as sodium tungstate dihydrate. Other catalysts that may be used include (NH₄)₆Mo₇O₂₄, CH₃ReO₄₅ and RuCl₃.

In a preferred embodiment, a reducing agent, such as sodium bisulfite, is added to the reaction mixture when the oxidation reaction has been completed. Alternative reducing agents may be concedered, e.g. SO₂, Na₂SO₃, Na₂S₂O₅.

In a preferred embodiment, the reaction mixture is neutralized by adding an alkaline compound such as NaOH or KOH after adding said reducing agent.

In a third aspect, the invention provides intermediate compounds according to formula I

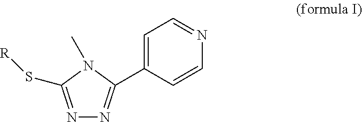

wherein
R is selected from the group of C₂₋₆alkyl, and C₅₋₁₀aryl-C₁₋₆alkyl,

Preferred such intermediate compounds are 4-Methyl-3-ethylthio-5-(4-pyridinyl)-1,2,4-triazole, and 4-Methyl-3-benzylthio-5-(4-pyridinyl)-1,2,4-triazole.

### Experimental Part

All starting materials are commercially available or earlier described in the literature.

By “room temperature” is meant (unless otherwise stated) a temperature in the range of 16-26° C.

### Example 1

### Preparation of 4-methyl-3-methylthio-5-(4-pyridinyl)-1,2,4-triazole

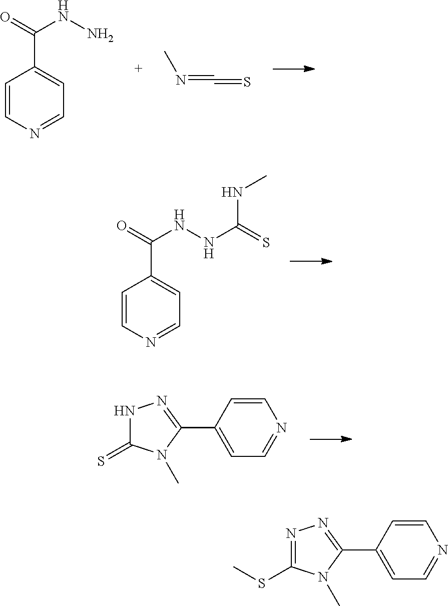

Water (1600 ml) was charged to a 2 L reaction vessel followed by isonicotinic acid hydrazide (201.15 g, 1.45 mol). An additional 50 ml of water was used to wash in isonicotinic acid hydrazide which had stuck to the addition funnel. The mixture was stirred at room temperature for fifteen minutes giving a clear solution. Methyl isothiocyanate (107.19 g, 1.42 mol) was charged as a solid in one portion. The solution phase of the resulting mixture became yellow. The mixture was warmed to 50° C. with stirring. After two and a half hours at this temperature, a thick, white slurry had formed and a solution of sodium hydroxide (66.1 g, 1.65 mol) in water (80 ml) was charged at 50° C., resulting in dissolution of the solids to give a clear, light-yellow solution. After 2 hours, the solution was cooled to 23° C. and iodomethane (228 g, 1.59 mol) was charged in one portion. An exotherm was noted with the temperature rising to 29° C. and the solution became a darker yellow colour. After 15 minutes, a very thick slurry was obtained and this was heated to 60° C. giving a clear orange-yellow solution which was cooled to 45° C. When the solution reached 48° C., seed crystals of 1-methyl-2-ethylthio-5-(4-pyridinyl)-1,3,4-triazole (0.47 g, 2.26 mmol) were charged and the resulting suspension cooled to 5° C. When the mixture had been at 5° C. for 50 minutes, the solids were collected by filtration on a glass filter, the filter-cake was washed with water (3×500 ml) and dried in a vacuum oven at 40° C. for 42 h giving 4-methyl-3-methylthio-5-(4-pyridinyl)-1,2,4-triazole as a white solid (264.0 g, assay 95.4% w/w, yield 86% based on methylisothiocyanate, contains 5.5% w/w of water).

### Example 2

### Preparation of 4-Methyl-3-ethylthio-5-(4-pyridinyl)-1,2,4-triazole

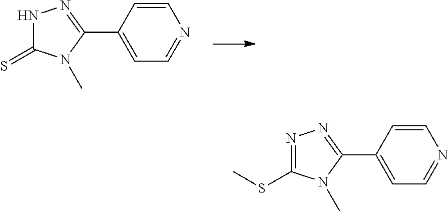

A mixture of 4-methyl-5-(4-pyridinyl)-1,2,4-triazolin-3-thione (5.22 g, 27.15 mmol) (obtained as an intermediate product in Example 1, sodium hydroxide (1.22 g, 30.5 mmol) and water (60 ml) was stirred at room temperature giving a cloudy, yellow solution. A is solution of iodo-ethane (4.68 g, 29.41 mmol) in acetone (2.5 ml) was charged and the clear, lemon-yellow solution stirred at room temperature for one hour. More acetone (20 ml) was then charged. After 21 hours, the solution was concentrated giving a sticky, light-orange solid which was triturated with acetone. The acetone solution was concentrated and the resulant solid was then triturated with dichloromethane. Evaporation of the dichloromethane solution gave the title product as a yellow solid (5.6 g, 93%)

### Example 3

### Preparation of 4-Methyl-3-benzylthio-5-(4-pyridinyl)-1,2,4-triazole

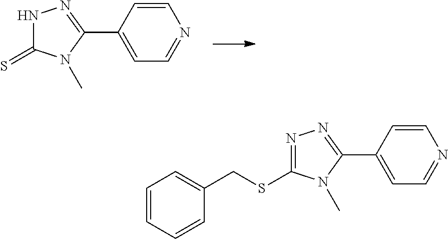

A mixture of 4-methyl-5-(4-pyridinyl)-1,2,4-triazolin-3-thione (5.37 g, 27.93 mmol) (obtained as an intermediate product in Example 1), sodium hydroxide (1.35 g, 33.75 mmol) and water (60 ml) was stirred at room temperature giving a cloudy, yellow solution. A solution of benzyl bromide (4.49 g, 28.18 mmol) in acetone (50 ml) was charged and the for ten minutes. Acetone was stripped under reduced pressure and a red solid precipitated giving a thick mixture which was diluted with water (50 ml). The solids were collected on a glass filter and the filter-cake was washed with water (50 ml) then dried under vacuum at 40° C. The title product was obtained as a red solid (7.1 g, assay 91% w/w, 82% yield).

### Example 4

### Preparation of 4-(5-Methanesulfonyl-4-methyl-4H-[1,2,4]triazol-3-yl)-pyridine

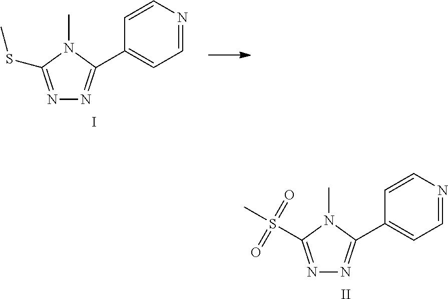

20 g (95.5 mmol) 4-(4-Methyl-5-methylsulfanyl-4H-[1,2,4]triazol-3-yl-pyridine (I) was charged to a 250 mL reactor. 60 mL water pre-mixed with 5.2 mL (95.5 mmol) sulfuric acid was added to the reactor and the temperature was set to 50° C. 321 mg (963.4 μmol) sodium tungstate Dihydrate was added to the solution in one portion followed by addition of 17.45 (203.8 mmol) hydrogen peroxide over 2 h. The solution was kept under stirring until completion. 6.7 g (19.1 mmol) sodium bisulfate was then added to quench excess peroxide. The pH was further adjusted to 3-4 by addition of 45% NaOH (aq), 9.1 mL was required. A thick precipitation was formed. The temperature was adjusted to 95° C., which resulted in dissolution of the precipitate. Compound II was then crystallized by applying a slow temperature gradient from 95-5° C. The crystals were finally filtered off, washed with 3×40 mL cold water and dried at 50° C. under reduced pressure. 17.3 g pure product corresponding to an isolated yield of 80% was achieved.

### Example 5

### Preparation of 4-(5-Methanesulfonyl-4-methyl-4H-[1,2,4]triazol-3-yl)-pyridine (NaMnO₄ method)

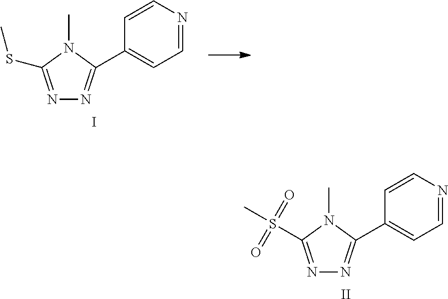

200 g (921.12 mmol) 4-(4-Methyl-5-methylsulfanyl-4H-[1,2,4]triazol-3-yl-pyridine (I) was dissolved in 860 mL acetic acid in a 2 L reactor. To the solution, 400 mL water was added. The temperature of the reaction was adjusted to below 10° C. 307 mL (1.2 mol) sodium permanganate (40% solution in water) was added drop-vise. The charge was exothermic and the temperature of the reaction mixture was kept around 20° C. throughout the addition. After completion of the reaction, 345 g sodiumsulfite dissolved in 1 L H₂O was the added to quench excess permanganate and produced MnO₂. The pH of the mixture was then adjusted to ˜6.3 by addition of NaOH (45% solution in water). During addition, is compound II precipitated. The crude product was filtered off and then suspended in 800 mL water. The mixture was stirred for 1 h and then filtered off and washed with 400 mL water. The product was further dissolved in 1.6 L acetonitrile at 65° C. and clear-filtered. The temperature was further adjusted to <10° C. to initiate crystallization. The acetonitrile was partially distilled of after which 1.6 L isopropylacetate was added. The remaining acetonitrile was then distilled off and the crystals were isolated by filtration. The cake was washed with 600 mL isopropylacetate. Finally, the crystals were dried at 50° C. under reduced pressure. 179.1 g product corresponding to an isolated yield of 82% was achieved.

1. A method of manufacturing a compound according to formula I: 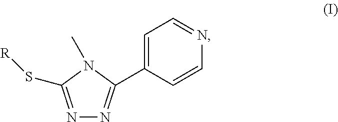
wherein:
R is C₁₋₆alkyl or C₅₋₁₀aryl-C₁₋₆alkyl; and
the method comprises:
a) reacting isonicotinohydrazide and methyl isothiocyanate, thereby obtaining 2-isonicotinoyl-N-methylhydrazinecarbothioamide;
b) under alkaline conditions, allowing 2-isonicotinoyl-N-methylhydrazinecarbothioamide to undergo a ring-forming reaction, thereby obtaining 4-methyl-5-pyridin-4-yl-2,4-dihydro-3H-1,2,4-triazole-3-thione; and
c) under alkaline conditions, allowing 4-methyl-5-pyridin-4-yl-2,4-dihydro-3H-1,2,4-triazole-3-thione to react with R—X, wherein R has the same meaning as in formula I and X is selected from the group of Cl, Br and I, thereby obtaining a compound according to formula I;
wherein steps a, b, and c) are carried out in an aqueous environment without intermediate isolations.

2. A method according to claim 1, wherein a single base selected from the group of NaOH, KOH, and amine bases is added in step b.

3. A method according to claim 1, wherein the compound of formula I obtained in step c is isolated by filtration.

4. A method for manufacturing a compound according to formula II: 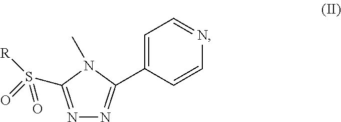
wherein:
R is C₁₋₆alkyl or C₅₋₁₀aryl-C₁₋₆alkyl; and
the method compises:
i) carrying out the method of claim 1; and
ii) oxidizing said compound according to formula I, thereby obtaining a compound according to formula II.

5. A method according to claim 4, wherein step ii is carried out in an acid aqueous solution of an oxidant selected from the group of hydrogen peroxide, sodium permanganate, potassium permanganate, NaIO₄, KIO₄, potassium monopersulfate, NaBO₃ and KBO₃.

6. A method according to claim 4, wherein step ii is carried out in an aqueous sulfuric acid solution.

7. A method according to claim 4, wherein step ii is carried out in presence of a catalytic amount of a tungstate, (NH₄)₆Mo₇O₂₄, CH₃ReO₃, or RUCl₃.

8. A method according to claim 4, wherein a reducing agent is added to the reaction mixture when the oxidation reaction of step ii has completed.

9. A method according to claim 8, wherein the reaction mixture is neutralized by adding an alkaline compound after adding reducing agent.
